(19) United States
(12) Patent Application Publication
(10) Pub. No.: US 2025/0186117 A1
(43) Pub. Date: Jun. 12, 2025

(54) DEVICE FOR REMOVING HAIR COLOR

(71) Applicant: Sophoxy LLC, Campbell, CA (US)

(72) Inventors: Benjamin Michael Fox, Campbell, CA (US); Douglas Alan Beumeler, San Jose, CA (US); Sophia Francisca Valentim, Campbell, CA (US)

(21) Appl. No.: 18/535,978

(22) Filed: Dec. 11, 2023

(51) Int. Cl. A61B 18/20; A61B 18/00

(52) U.S. Cl. CPC A61B 18/203; A61B 2018/00476

(57) ABSTRACT

A device includes a first arm, a second arm, a laser beam source, and an optical medium. The first arm is pivotably attached to the second arm and the first arm and the second arm move respective to one another. The laser beam source is configured to generate laser beam that is between 400-800 nm in wavelength. The optical medium is positioned on the second arm and configured to receive the generated laser beam and apply the generated laser beam to hair strands placed between the first arm and the second arm when the first arm and the second arm are in closed position.

### BACKGROUND

The current leading method of cosmetic pigmentation removal in hair is to apply a series of chemicals (commonly referred to as bleaching) to the hair shaft which chemically opens the shaft and oxidizes the color compounds. In some conventional methods, removing hair color may be through forced oxidation of pigment compounds called melanin using hydrogen peroxide. Melanin is typically located throughout the interior of the hair shaft in the cortex layer, thereby requiring the bleaching compound to be applied for a significant amount of time. Unfortunately, oxidation is not a selective chemical process and cannot be isolated to the color compounds and reacts with the other molecules in hair resulting in permanent mechanical damage to the hair fiber (e.g., keratin). For example, it is common for hair structure to be damaged and become fragile, break, and end up with poor texture. Additionally, the chemicals used in the bleaching process may adversely react with certain types of skin if exposure occurs.

### SUMMARY

Accordingly, a need has arisen to lighten hair (e.g., remove color) with reduced damage to hair structure and/or skin if exposed. In some embodiments, a laser is used to target one or both types of melanin in hair with high enough energy to destroy the melanin molecules without damaging hair structure and/or impacting skin. In some embodiments, the amount of laser (i.e., amount of energy) delivered to the hair impacts the amount of melanin or dye compounds that will be destroyed, thereby controlling the amount that hair will lighten.

According to some embodiments, a laser pulse within a wavelength range is used and selected to deliver enough energy to destroy the melanin of hair without impacting the hair structure or the skin. In one embodiment, a laser device is integrated within a clamping device to deliver a laser beam when in closed position and remove hair color. It is appreciated that the device when in open position does not deliver a laser beam for safety reasons. As the hair passes through the clamping device, the laser pulses activate, which are primarily absorbed by the melanin molecules effectively destroying the pigmentation and removing color.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 depicts an example of a handheld apparatus for removing hair color according to one aspect of the present embodiments.

FIG. 2 depicts an example of a flat iron with a laser source for removing hair color according to one aspect of the present embodiments.

FIG. 3 depicts an example of absorption of energy versus wavelength of a laser beam.

FIG. 4 depicts an example of melanin absorption within a visible light wavelength according to one aspect of the present embodiments.

FIG. 5 depicts an example of absorption coefficient versus wavelength according to one aspect of the present embodiments.

FIG. 6 depicts an example of synchronized pulse to remove hair color according to one aspect of the present embodiments.

FIG. 7 depicts an example of a laser source and shaper according to one aspect of the present embodiments.

### DETAILED DESCRIPTION

The following disclosure provides many different embodiments, or examples, for implementing different features of the subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Before various embodiments are described in greater detail, it should be understood that the embodiments are not limiting, as elements in such embodiments may vary. It should likewise be understood that a particular embodiment described and/or illustrated herein has elements which may be readily separated from the particular embodiment and optionally combined with any of several other embodiments or substituted for elements in any of several other embodiments described herein. It should also be understood that the terminology used herein is for the purpose of describing the certain concepts, and the terminology is not intended to be limiting. Unless defined otherwise, all technical and scientific terms used herein have the same meaning as commonly understood in the art to which the embodiments pertain.

As described above, there is a need to lighten hair (e.g., remove color) with minimal damage to hair structure and/or skin if exposed. In some embodiments, a laser is used to target one or both types of melanin in hair with sufficient enough energy to destroy the melanin molecules without damaging hair structure and/or impacting skin. In some embodiments, the amount of power (i.e., amount of energy) delivered to the hair impacts the amount of melanin that will be destroyed, thereby controlling the amount that hair will lighten.

According to some embodiments, a laser pulse within a wavelength range is used and selected to deliver enough energy to destroy the melanin of hair while reducing damage to hair structure or the skin. In one embodiment, a laser device is integrated within a clamping device to deliver laser beam when in closed position and remove hair color. It is appreciated that the device when in open position does not activate and deliver the laser beam for safety reasons. As the hair passes through the clamping device, the laser pulses activate, which is primarily absorbed by melanin effectively destroying the color molecules and removing the color.

In general, there are two primary melanin molecules which create the pigmentation in mammalian hair. One is pheomelanin (brown-black hues) and another is eumelanin (red-yellow hues). Due to the similarities between them, the use of the single term “melanin” throughout this application refers to both unless a specific molecule is being described in which case the full term is used. The melanin molecule evolved as protection from the damaging properties of solar radiation, particularly in the ultraviolet spectrum, which can cause cellular damage after extended exposure.

In hair, the melanin is produced only during the initial growth stages while hair is in the follicle and remains in that same concentration unless altered chemically or by prolonged exposure to higher energy levels of electromagnetic radiation. When melanin absorbs light, most of that energy is converted into heat. With significant heating, the molecule breaks some of its chemical bonds and is reduced to smaller molecules which no longer express the pigmentation hues. This process is often witnessed in individuals that spend significant time in the sun and their hair appears to have lightened in color. This is often referred to as “sun-bleached” and is the mechanism that will be leveraged. By using directed and concentrated light, such as a laser, and selecting wavelengths that minimize absorption in non-melanin molecules (specifically keratin), a device capable of reducing or removing the pigmentation in hair can be realized.

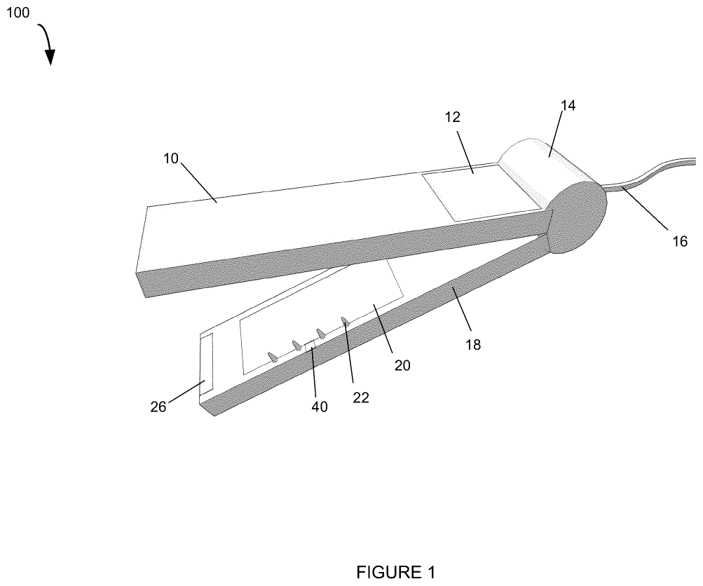

FIG. 1 depicts an example of a handheld apparatus for removing hair color in accordance with some embodiments. The device 100 includes a first arm 10 and a second arm 18 that are connected to one another via a hinge 14 and move (e.g., rotate about the hinge 14) with respect to one another. The device 100 may be powered via a power cable 16. It is appreciated that the device 100 may further include a laser beam source 40 that is configured to generate a laser beam and emit the generated laser beam via an optical medium 20. The optical medium 20 may be positioned on the first arm 10 and/or the second arm 18 or a combination thereof that when activated emits a laser beam to hair positioned within the first arm 10 and the second arm 18, thereby removing the hair color. It is appreciated that in some embodiments, the device 100 may further include a sensor 26 (e.g., optical sensor, capacitive sensor, MEMS sensor, etc.) configured to detect when the device 100 is in closed position and to detect when the device 100 is in open position. The device 100 may include a controller 12 configured to control the operation of the device 100. For example, in some embodiments, the controller 12 may control the wavelength of the laser being emitted, the pulse duration, the power, etc. In other words, the controller 12 is configured to change a characteristic of the generated beam based on the sensed data, e.g., color, motion, etc., from the sensor.

In some embodiments, the controller 12 may deactivate the optical medium 20 and/or the laser beam source 40 to prevent the laser beam from being emitted when the controller 12 in response to a signal from the sensor 26 determines that the device 100 is in open position. In yet one nonlimiting example, the controller 12 may activate the optical medium 20 and/or the laser beam source 40 to cause the laser beam to be emitted in response to a signal from the sensor 26 that the device 100 is in a closed position, thereby removing hair color. It is appreciated that activating/deactivating the optical medium 20 and/or the laser beam source 40 refers to any means to initiate the laser beam emission or preventing the laser beam emission. In some embodiments, activating/deactivating the laser beam from being emitted may be achieved via a variety of mechanisms, e.g., a switch to turn off the laser beam source. Detecting whether the device 100 is in open/close position serves as a safety feature to prevent a person from inadvertently damaging body, e.g., damaging eye from laser exposure, damaging skin from laser exposure, etc.

It is appreciated that in some embodiments, the device 100 may be a cosmetology device, e.g., flat iron, curling iron, etc., where the laser beam source 40 and/or the optical medium 20 are integrated within the hair straightener, curling iron, etc. It is appreciated that in some optional embodiments, the device 100 may further include a mechanical means 22 for distributing hair fibers.

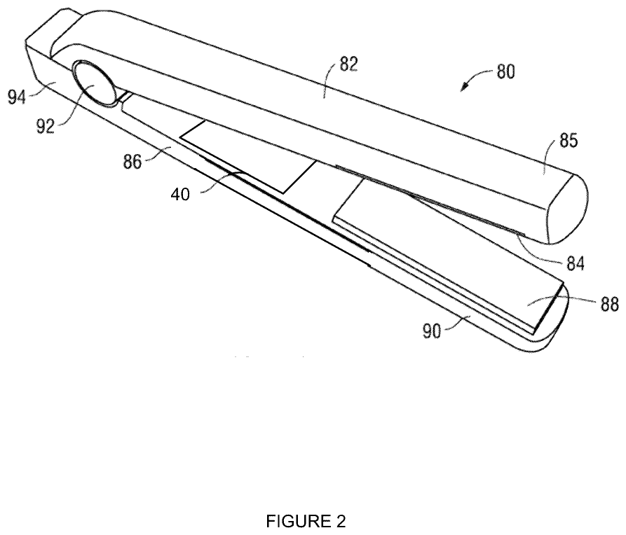

Referring now to FIG. 2, a nonlimiting example of the laser beam source 40 integrated within a flat iron is shown according to some aspects of the embodiments. In this example, the flat iron 80 has a top elongated arm 82 with a first heating element that is affixed inside the top elongated arm 82 and a first heating plate 84 at the distal end 86 of the top elongated arm 82. The first heating element is attached to the first heating plate 82. In some embodiments, the flat iron 80 also includes a bottom elongated arm 86 with a second heating element that is affixed inside the bottom elongated arm 86 and a second heating plate 88 at the distal end 90 of the bottom elongated arm 86. The second heating element is attached to the heating plate 88. The top and bottom elongated arms 82 and 86 are attached to each other via a pivot connection 92 at the proximal end 94 of the elongated arms. In some embodiments, a spring is affixed between the top and the bottom elongated arms 82 and 86 at the pivot connection 92. In one nonlimiting example, the spring is based to maintain the top elongated arm 82 and the bottom elongated arm 86 in an open position, requiring the user to squeeze the elongated arms toward one another to close and lock hair between the elongated arms 82 and 86 in order to bring the first and the second plates into proximity of one another, thereby directing heat into the one or more locks of hair that are held between the plates.

It is appreciated that the laser beam source 40 as described above may be integrated within a flat iron 80 or separately generated and carried to the device via optical cabling. The laser beam source 40 operates substantially similar to that of FIG. 1, described above. It is appreciated that various other components such as the controller 12, the optical medium 20, etc., are not illustrated in FIG. 2, for brevity but may be present, as described in FIG. 1. Accordingly, a single device may be used for flattening hair, curling, etc., in addition to removing hair color. It is appreciated that the laser beam source 40 integrated within a flat iron 80 is shown for illustrative purposes and should not be construed as limiting the scope of the embodiments. For example, the laser beam source 40 and its associated components may be integrated within other cosmetology devices.

It is appreciated that in some nonlimiting examples, the first heating plate 82 and the second heating plate 88 are aluminum plates that during application of color removal are used to cool the air by acting as a heat sink instead of a heat source. In some nonlimiting examples, a different cooling mechanism may be used to remove the heat generated by the laser source 40. For example, a waterline may be attached to the device in order to circulate water through the device in order to remove heat and act as a heat sink. In yet another example a waterline may be used to wet hair in order to remove heat from the hair when the laser source 40 applies the beam.

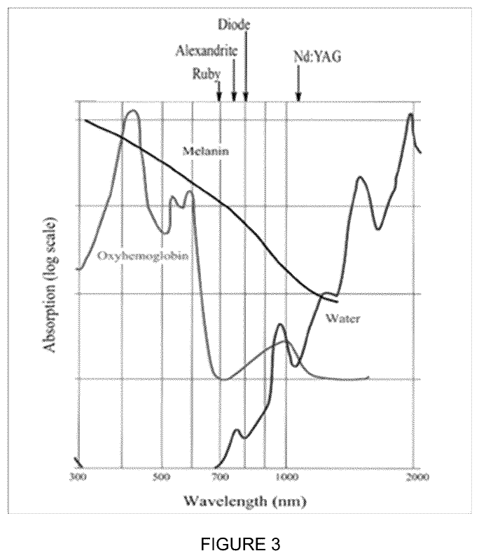

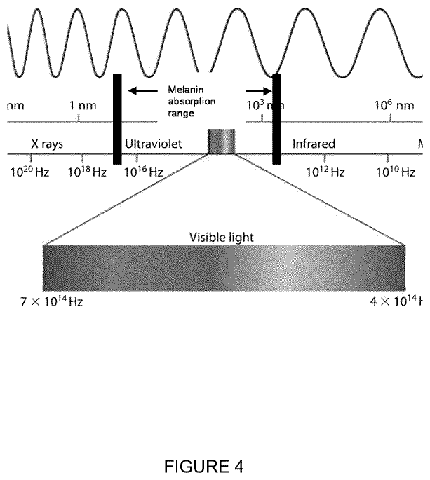

In general, melanin molecules are a protection mechanism, they absorb over a large band of wavelengths, as shown in FIG. 3, maximizing near the ultraviolet end of the spectrum 335 nm, and continuing into the infrared range of circa 1500 nm, as shown in FIG. 4. This large absorptive range allows for several options in the design of the laser beam source 40.

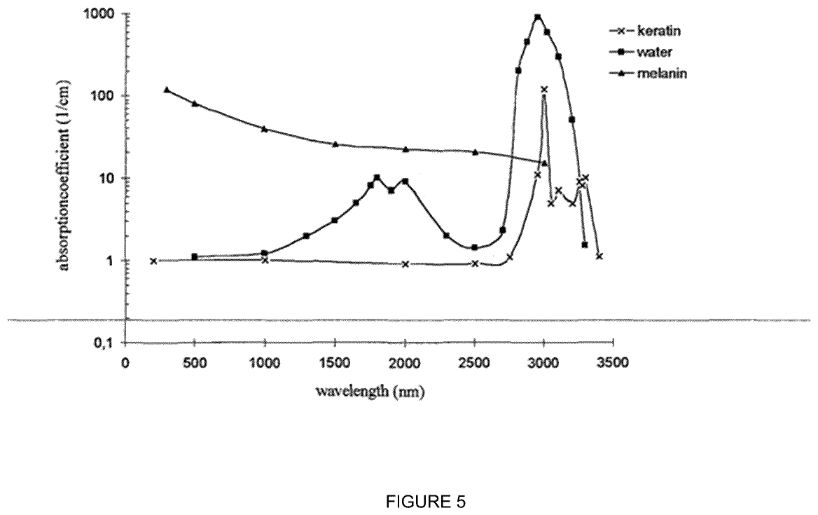

The smaller the wavelength, the higher the energy which increases penetration depth/level but also increases the likelihood of damage due to non-thermal mechanisms. Therefore, wavelengths of less than 400 nm in the ultraviolet spectrum are not suitable for the laser wavelength of the laser beam source 40. As the wavelength increases in size, more molecules are able to absorb the incoming electromagnetic radiation. At the infrared range of the spectrum, organic and metallic molecules increase significantly in their absorption. Additionally, water begins to absorb significantly in the infrared range. As a result, using wavelengths for the device significantly in the infrared spectrum unnecessarily generates excessive heat resulting in a high potential to pass the energy threshold for permanent damage to the hair structure as well as inefficient absorption by the targeted molecules, as shown in FIG. 5. Therefore, wavelengths over around 800 nm are also not suitable. As such, the laser beam source 40 is configured to generate laser beam between the wavelengths of 400-800 nm.

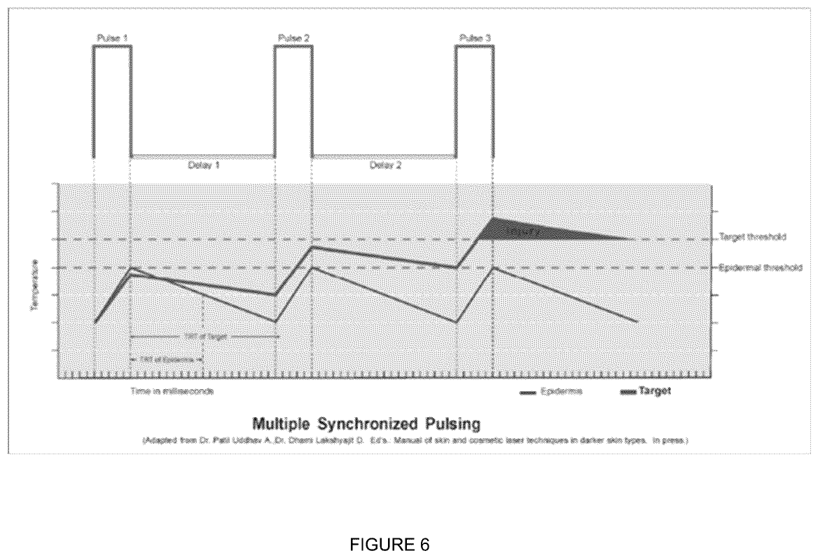

It is also appreciated that pulse duration and power are related. Accordingly, another factor that should be considered is pulse duration. In general, melanin requires a minimum amount of absorbed energy to break the chemical bonds, but also requires a very quick, short pulse to limit residual heating and therefore damage to the surrounding hair structure. The faster the pulse duration, the more energy is required in that pulse. A series of very fast (10-50 picosecond or 5-25 nanosecond duration) pulses can effectively push enough energy to the melanin without crossing the damage threshold for the unintended targets, as illustrated in FIG. 6. It is appreciated that while the series of very fast pulses may not be necessary for darker hair with significant melanin content (0.75-2%), it could be used on hair with very low pigmentation concentration (e.g., 1.1% for brown hair, 0.06% for Scandinavian blonde, etc.).

It is appreciated that as wavelengths approach and enter the infrared spectrum, much less energy is required before irreparable destruction occurs to the hair surface. The design criteria range of 400-800 nm is appropriate to effectively remove hair color while reducing risk of damage. Therefore, controller 12 may be used to configure the laser source beam 40 in order to deliver the right amount of energy (depending on how light or dark hair may be) to the hair bundles. The controller 12 in some examples, receives one or more input selection from a user (e.g., indicating hair color, hair type such as Caucasian or African American, etc.) and adjusts the laser beam source 40 accordingly that is most suitable for the indicated type and hair color. In some nonlimiting examples, a laser beam with a wavelength of 532 nm may be generated with a picosecond or nanosecond duration to deliver energy to hair coloring compounds (e.g. melanin). It is appreciated that in tattoo laser removal, a wavelength of approximately 532 nm has been used. As such, laser with wavelength of 532 nm may be used for both natural melanin removal (in hair) as well as from artificial dyes. In hair with lighter pigments such as blond hair no destruction of hair structure has been observed. In hair with significant melanin (e.g., black hair, etc.) the wavelength of 532 nm should deliver enough energy not to exceed approximately 28.5 J/cm² which is the amount of energy threshold that results in destruction of hair structure. In some nonlimiting examples, the laser beam with a wavelength of 577 nm may be used.

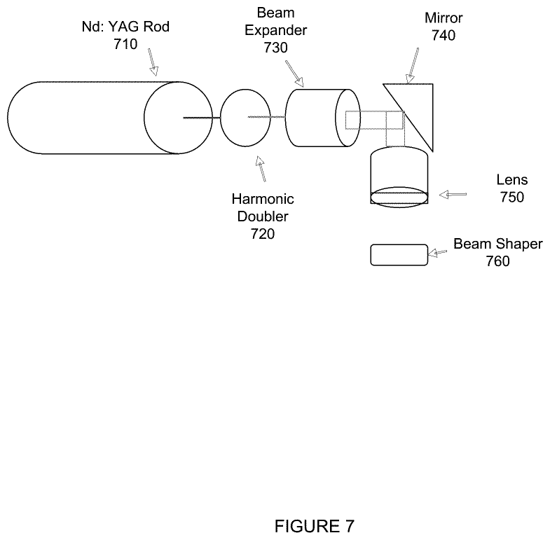

It is appreciated that generating a laser beam within a wavelength of 400-800 nm may be through generating a higher wavelength laser beam, e.g., 1064 nm, via Nd: YAG rod 710 (neodymium doped YAG) and passing the generated laser beam through a harmonic doubler 720, which is a frequency doubling medium to generate a second harmonic of 532 nm that is suitable for removing hair color. In some embodiments, a different doping molecules may result in a different wavelength. It is appreciated that other components may optionally be used, e.g., beam expander 730 to expand the laser beam (i.e., spreads the laser beam before it is reflected to the head of the instrument), mirror 740 to reflect the laser beam, lens 750 to focus/defocus the laser beam, and beam shaper (e.g. a Powell lens) 760 to shape the laser beam (generating a beam shape such as rectangular shape that may be 1 inch wide by ⅛ inch tall), as shown in FIG. 7. It is appreciated that the final beam size and shape may vary, as desired. It is further appreciated that the device may include various settings to change the power, the pulse duration, the shape, etc., depending on the type of hair being treated.

It is appreciated that applying the laser beam to every hair strand to achieve a uniform and consistent result may be desired. Since the outer layers of hair bundles have the tendency of masking deeper strands, using a material that is reflective at the proper device wavelength encourages less wasted light and more effective coverage. Accordingly, extruded comb made from reflective material such as Aluminum may be used to reflect the laser beam to achieve consistent and uniform results. The height of the comb sets the focal length and vertical bundle vertical quantity, the width sets the bundle width, the material sets the reflection or absorption of unused light, and the geometry sets the reflectance angle of that light back toward the bundles.). In some nonlimiting examples, the comb may be a few square inches (e.g., 1 square inch) comprising an aluminum plate with V-shaped and/or U-shaped grooves that are positioned under the beam and align the direct that the hair is being pulled through.

The foregoing description of various embodiments of the claimed subject matter has been provided for the purposes of illustration and description. It is not intended to be exhaustive or to limit the claimed subject matter to the precise forms disclosed. Many modifications and variations will be apparent to the practitioner skilled in the art. Embodiments were chosen and described in order to best describe the principles of the invention and its practical application, thereby enabling others skilled in the relevant art to understand the claimed subject matter, the various embodiments and the various modifications that are suited to the particular use contemplated.

What is claimed is:

1. A device comprising:
a first arm;
a second arm, wherein the first arm is pivotably attached to the second arm and wherein the first arm and the second arm move respective to one another;
a laser beam source configured to generate laser beam that is between 400-800 nm in wavelength; and
an optical medium positioned on the second arm, wherein the optical medium is configured to receive the generated laser beam and apply the generated laser beam to hair strands placed between the first arm and the second arm when the first arm and the second arm are in closed position.

2. The device of claim 1 further comprising a sensor configured to generate a signal in response to the first arm and the second arm moving into the closed position.

3. The device of claim 2 further comprising a controller configured to receive the generated signal and in response to determining that the first arm and the second arm are in the closed positioned activates the laser beam source to generate the laser beam.

4. The device of claim 3, wherein the controller is configured to receive an input from a user to adjust power of the laser beam.

5. The device of claim 3, wherein the controller is configured to adjust a pulse duration for the generated wavelength.

6. The device of claim 1, wherein the first arm and the second arm are in the closed position when the first arm and the second arm are squeezed toward one another to lock hair strands in between the first arm and the second arm.

7. The device of claim 1, wherein the generated laser beam has a pulse duration between 10 picoseconds and 50 nanoseconds.

8. The device of claim 1 further comprising a power cable to supply power to the laser beam source.

9. The device of claim 1 further comprising mechanical means positioned on the first arm or the second arm, wherein the mechanical means is configured to distribute hair strands when hair strands are positioned between the first arm and the second arm.

10. The device of claim 1 further comprising a harmonic doubler configured to generate wavelength harmonics for the generated laser beam.

11. The device of claim 1 further comprising a beam expander configured to spread the generated laser beam before it is reflected out from the optical medium and before it is applied to hair strands.

12. The device of claim 1 further comprising a lens configured to adjust the focus the generated laser beam before it is applied to hair strands.

13. The device of claim 1 further comprising a beam shaping lense configured to generate a beam shape for the generated laser beam before it is applied to hair strands.

14. The device of claim 13, wherein the beam shape is rectangular.

15. A flat iron comprising:
a top elongated arm with a first heating element attached thereto, and wherein the top elongate arm includes a first heating plate to apply the generated heat to hair strands when the hair strands are locked between the top elongated arm and a bottom elongated arm;
the bottom elongated arm with a second heating element attached thereto, and wherein the bottom elongate arm includes a second heating plate to apply the generated heat to hair strands when the hair strands are locked between the top elongated arm and the bottom elongated arm;
a laser beam source configured to generate laser beam that is between 400-800 nm in wavelength; and
an optical medium positioned on the top elongated arm and positioned in close proximity to the first heating plate, wherein the optical medium is configured to receive the generated laser beam and apply the generated laser beam to hair strands placed between the top elongated arm and the bottom elongated arm when the top elongated arm and the bottom elongated arm are in closed position.

16. The flat iron of claim 15 further comprising a sensor configured to generate a signal in response to the top elongated arm and the bottom elongated arm moving into the closed position.

17. The flat iron of claim 16 further comprising a controller configured to receive the generated signal and in response to determining that the top elongated arm and the bottom elongated arm are in the closed positioned activates the laser beam source to generate the laser beam.

18. The flat iron of claim 17, wherein the controller is configured to receive an input from a user to adjust a wavelength of the laser beam.

19. The flat iron of claim 17, wherein the controller is configured to adjust a pulse duration for the generated wavelength.

20. The flat iron of claim 15, wherein the top elongated arm and the bottom elongated arm are in the closed position when the top elongated arm and the bottom elongated arm are squeezed toward one another to lock hair strands in between the top elongated arm and the bottom elongated arm.

21. The flat iron of claim 15, further comprising a sensor and a controller coupled to one another, wherein the controller receives one or more sensed data from the sensor and wherein the controller is configured to adjust a characteristic of the generated laser beam based on the sensed data.

22. The flat iron of claim 15 further comprising a cooling mechanism configured to remove heat generated by the laser beam source.
